(19) United States
(12) Patent Application Publication
(10) Pub. No.: US 2014/0158562 A1
(43) Pub. Date: Jun. 12, 2014

(54) PACKAGED DIANHYDROHEXITOLS HAVING HIGH CHEMICAL STABILITY

(71) Applicants: Mathias Ibert, La Chapelle D'armentieres (FR); Herve Wyart, Cuinchy (FR)

(72) Inventors: Mathias Ibert, La Chapelle D'armentieres (FR); Herve Wyart, Cuinchy (FR)

(73) Assignee: ROQUETTE FRERES, Lestrem (FR)

(21) Appl. No.: 14/236,970

(22) PCT Filed: Aug. 3, 2012
(86) PCT No.: PCT/FR2012/051842

§ 371 Date: Feb. 4, 2014

(30) Foreign Application Priority Data
Aug. 5, 2011 (FR) 1157209

(51) Int. Cl. A61K 47/02; B65B 31/02; C07D 493/04

(52) U.S. Cl. CPC A61K 47/02; C07D 493/04; B65B 31/02
USPC 206/213.1; 53/432

(57) ABSTRACT

A dianhydrohexitol packaged in a packaging material that is impervious to gas, wherein the partial pressure of oxygen inside the packaging is 0.1 to 10 mbar, preferably 0.5 to 5 mbar, and more preferably 0.5 to 2 mbar. The method for packaging dianhydrohexitol, includes inserting the dianhydrohexitol into a packaging material that is impervious to gas, and then hermetically sealing the packaging, the method being carried out in an atmosphere that has a partial pressure of oxygen of 0.1 to 10 mbar, preferably 0.5 to 5 mbar, and more preferably 0.5 to 2 mbar.

### FIELD OF THE INVENTION

The present invention relates to dianhydrohexitols packaged in such a way that their chemical stability is retained even after long-term storage. The dianhydrohexitols packaged according to the invention also exhibit the distinguishing feature of not being subject to compacting.

### PRIOR ART

Dianhydrohexitols, also known as isohexides, are products of the internal dehydration of hydrogenated C₆ sugars (hexitols), such as sorbitol, mannitol and iditol.

Among these doubly dehydrated hydrogenated sugars, isosorbide is today that for which the most industrial applications are being developed and are being envisaged to be developed, in particular in the pharmaceutical field, in the field of chemical synthesis intermediates and in the field of plastics.

It is a relatively recent observation that dianhydrohexitols and in particular isosorbide are products which are chemically not very stable.

The applicant company has in particular observed that the storage of dianhydrohexitols, in particular of isosorbide, even with the exclusion of atmospheric moisture, can result in a chemical decomposition resulting, inter alia, in the formation of formic acid, which acid exhibits a characteristic unpleasant odor particularly troublesome in pharmaceutical applications. Furthermore, this acid causes problems of coloration during the synthesis of polymers.

The applicant company has thus been prompted to develop processes for the purification and stabilization of dianhydrohexitols described in particular in the patent applications EP 1 287 000 and WO 03/043959.

However, the applicant company subsequently noticed that the shelf lives of dianhydrohexitols, determined under the conditions of the stability tests described in the applications EP 1 287 000 and WO 03/043959, only imperfectly reflected the stability of these same products under actual conditions of transportation and storage. The applicant company has in particular recorded, in some cases, relatively higher concentrations of formic acid close to the polyethylene packaging film. This locally high concentration could give rise to the thought that the decomposition of dianhydrohexitols and in particular isosorbide does not take place only according to intrinsic temperature-dependent kinetics but is also related, inter alia, to the interaction with the packaging material.

The applicant company thus set itself the aim of finding a relatively inexpensive packaging which makes it possible to package dianhydrohexitol compositions and which, in contrast to the polyethylene conventionally used under standard conditions, makes possible very-long-term storage without damaging the chemical stability, even close to the packaging material.

Patent application JP 2006-117649 discloses the use of a packaging material of film type for packaging isosorbide with the aim of protecting the latter from absorption of water, of keeping it in the fluid powder form and of preventing the formation of aggregates. The packaging film is defined very vaguely as being a multilayer film. In addition, this document does not mention the problem of the chemical instability of dianhydrohexitols on contact with the packaging material.

The applicant company itself developed a specific packaging based on thermoplastic polymer comprising at least 0.1% of at least one antioxidant for packaging dianhydrohexitols. The applicant company then tested, in the patent application WO 2009/019371, various sachets composed of various packaging materials. The test consisted in introducing 50 g of isosorbide into the test sachets, in immediately and hermetically closing said sachets and in then placing the closed sachets in a second sachet made of aluminum comprising a polyethylene coating and also closed by welding, in order to ensure leaktightness with respect to the external atmosphere. Only the specific packaging comprising at least 0.1% of at least one antioxidant made it possible to prevent chemical decomposition of the dianhydrohexitol compositions during their storage at a temperature of 50° C. for a period of time of greater than 1.5 months.

However, such technical solutions require the preparation of specific packaging materials and leave unresolved the problem of the compacting of the dianhydrohexitols during their storage in bags of several kilograms, indeed even of several tens of kilograms.

Furthermore, this type of packaging is not suitable for the liquid forms of isosorbide.

The main aim of the present invention is thus to provide a simple packaging for dianhydrohexitols which does not require the preparation of complex packaging materials and which makes it possible, however, to retain the chemical stability of dianhydrohexitols in whatever form, this being the case even for storage for long periods of time and/or at high temperatures, i.e. storage for a period of time of greater than 2 months at a temperature of at least 50° C.

Another specific aim of the present invention consists in providing packaged dianhydrohexitols which do not exhibit compacting, even after storage for several weeks and/or storage at high temperatures.

Another specific aim of the present invention is also to provide dianhydrohexitol compositions which can easily flow from the outlet of their packaging and which leave no or virtually no residues in the emptied packaging sachet after use.

### SUMMARY OF THE INVENTION

A subject matter of the present invention is consequently a dianhydrohexitol packaged in a gastight packaging material, characterized in that the oxygen O₂ partial pressure inside the packaging is between 0.1 mbar and 10 mbar, preferably between 0.5 mbar and 5 mbar and more preferably still between 0.5 mbar and 2 mbar.

An additional subject matter of the invention is a process for packaging dianhydrohexitol comprising the introduction of said dianhydrohexitol into a gastight packaging material and then the hermetic closing of said packaging, said process being characterized in that it is carried out in an atmosphere exhibiting an oxygen partial pressure of between 0.1 mbar and 10 mbar, preferably between 0.5 mbar and 5 mbar and more preferably still between 0.5 mbar and 2 mbar.

### DETAILED DESCRIPTION

Dianhydrohexitols (1,4:3,6-dianhydrohexitols) comprise in particular isosorbide (1,4:3,6-dianhydrosorbitol), isomannide (1,4:3,6-dianhydromannitol), isoidide (1,4:3,6-dianhydroiditol) and the mixtures of at least two of these products. Preferably, the packaged dianhydrohexitol according to the present invention comprises isosorbide or is essentially composed of isosorbide. It is preferably a composition for which the isosorbide content is at least equal to 95% by weight, more preferably at least equal to 97% by weight and more preferably still at least equal to 98.5% by weight (dry/dry).

The dianhydrohexitols according to the invention can in particular result from any preparation process known to a person skilled in the art, for example from a distillation of a crude reaction product, from a product purified by melt crystallization or crystallization in an aqueous or organic solvent, optionally followed by melting, from a concentration to dryness of a purified solution of dianhydrohexitols according to the patent EP 1 287 000, and the like.

The present invention applies to both solid and liquid dianhydrohexitol compositions.

As solid forms, they can, for example, be cooled and solidified distillates or be crystals, it being possible for all of these products in particular to be provided in the form of a powder, of crystals, of flakes or of pellets. Preferably, the packaged dianhydrohexitol according to the invention is in the form of a powder, of crystals, of flakes or of pellets.

As liquid forms, they can, for example, be isosorbide melts, that is to say isosorbide of high purity, 98% to 100% by weight, maintained in the molten form at a temperature of greater than or equal to 63±2° C. (at atmospheric pressure), or be isosorbide in solution, in particular in solution in water or in an organic solvent, in a proportion in particular of 50% to 90% by weight, preferably of 65% to 85% by weight, of dissolved isosorbide.

As was explained above, dianhydrohexitols generally exhibit a high chemical instability, in particular on contact with their packaging material, when they are stored over long periods of time and at high temperature.

Many technical solutions have been envisaged but the latter always required the preparation of specific packaging materials and furthermore left unresolved the problem of compacting of the dianhydrohexitols during their storage.

During its many research studies, the applicant company devised a vacuum packaging for dianhydrohexitols. It was interested in particular in the various pressure ranges conventionally used in industry, in particular in the food-processing and chemical industries.

In the present patent application, the term “vacuum” is used to denote an absolute pressure in a given chamber which is less than atmospheric pressure, that is to say less than approximately 1013 mbar.

In the present invention, the pressures, both absolute and relative, are evaluated according to any technique well known to a person skilled in the art and at sea level. The absolute pressure, thus evaluated at sea level, is approximately 1013 mbar. A person skilled in the art will know how to adjust the pressure levels of the present invention according to the altitude and the conditions.

As a reminder, the partial pressure of one of the gases making up a gas mixture is equal to the percentage of this gas in the mixture multiplied by the absolute pressure of the mixture. Thus, the sum of the partial pressures is equal to the absolute pressure.

P gas=AP×% gas
P gas: partial pressure of the gas in the gas mixture
AP: absolute pressure (for example, at sea level, 1013 mbar)
% gas: percentage of the gas in the gas mixture

The applicant company initially assumed that a reduction in the oxygen O₂ partial pressure inside the dianhydrohexitol packaging was liable to increase the stability of said dianhydrohexitols. However, the applicant company has demonstrated, by way of example, that, in order to make possible both a packaging which can be envisaged industrially and an actual maintenance of the chemical stability, very low partial pressures, i.e. of the order of 0.01 to 0.001 mbar, or, on the contrary, relatively high partial pressures, i.e. of the order of 50 to 100 mbar, are not at all acceptable.

One of the technical solutions which can be envisaged for reducing the oxygen partial pressure inside a hermetically closed chamber consists in establishing a vacuum in said chamber and thus an absolute pressure less than approximately 1013 mbar.

In the present invention, the applicant company has been interested only in pressure ranges:
which are industrially acceptable, that is to say not requiring a lengthy packaging time and/or extremely delicate and expensive equipment, as is the case in order to obtain an oxygen partial pressure inside the packaging in particular of less than 0.1 mbar;
which make possible an actual chemical stability of the dianhydrohexitol, this not being the case with high oxygen partial pressures, in particular of greater than 10 mbar.

The applicant company has noticed that a reduction in the oxygen partial pressure of the order of 50% (oxygen partial pressure of approximately 100 mbar inside the packaging) in the packaging, just like a reduction of the order of 75% (oxygen partial pressure of approximately 50 mbar inside the packaging), has no effect on the stability of the dianhydrohexitols.

Likewise, filling the packagings with dianhydrohexitols while continuously flushing with nitrogen N₂ did not make it possible to retain the chemical stability of their dianhydrohexitols over a long period of time.

Finally, it is entirely by chance, during an additional test relating to packagings which are hermetic toward oxygen, that the applicant company noticed that there exists an oxygen partial pressure threshold inside the dianhydrohexitol packaging below which the dianhydrohexitols exhibit a chemical stability which is retained during long term storage and/or storage under high temperatures.

Furthermore and surprisingly, the applicant company has demonstrated that dianhydrohexitols can be stored in any packaging material provided that it is gastight and that the atmosphere inside the packaging exhibits a very particular oxygen partial pressure, in particular of less than or equal to 10 mbar, preferably of less than or equal to 5 mbar and more preferably still of less than or equal to 2 mbar. Unexpectedly, such a packaging form also solves the problem of the chemical instability of dianhydrohexitols on contact with the packaging material.

In the present invention, the term “gastight” is used to denote the packagings for which the permeability to oxygen is less than or equal to 0.5 cm³/m²/24 hours, preferably less than or equal to 0.2 cm³/m²/24 hours (measures carried out at 23° C. and 0% residual moisture according to the standard ASTM 3985).

The subject matter of the present invention is consequently a dianhydrohexitol packaged in a gastight packaging material, characterized in that the oxygen O₂ partial pressure inside the packaging is between 0.1 mbar and 10 mbar, preferably between 0.5 mbar and 5 mbar and more preferably still between 0.5 mbar and 2 mbar. In the present invention, the expression “between X and Y” does not exclude said limits X and Y.

An appropriate reduction in the oxygen partial pressure of the atmosphere inside the dianhydrohexitol packaging can in particular be obtained by virtue of a packaging of said dianhydrohexitols under vacuum.

Another subject matter of the present invention is thus a packaged dianhydrohexitol, characterized in that:
the atmosphere inside the packaging exhibits an oxygen O₂ partial pressure of between 0.1 mbar and 10 mbar, preferably of between 0.5 mbar and 5 mbar and more preferably still of between 0.5 mbar and 2 mbar, and
the absolute pressure inside the hermetically closed packaging is between 0.5 mbar and 50 mbar, preferably between 2 mbar and 25 mbar and more preferably still between 2 mbar and 10 mbar.

The applicant company has also sought to solve the two-fold problem posed by dianhydrohexitols, namely the problem of their very poor chemical stability and also the problem of their high propensity to compact (or cake). Thus, another subject matter of the present invention is a packaged dianhydrohexitol, characterized in that the atmosphere inside the packaging exhibits, in addition to a reduced oxygen partial pressure, an absolute pressure inside the hermetically closed packaging of greater than or equal to 250 mbar, preferably of greater than or equal to 400 mbar and more preferably still of greater than or equal to 500 mbar.

Another subject matter of the present invention is a packaged dianhydrohexitol, characterized in that the atmosphere inside the packaging exhibits a reduced oxygen partial pressure, an absolute pressure inside the hermetically closed packaging of greater than or equal to 250 mbar and a nitrogen partial pressure of between 240 mbar and 1012.9 mbar, preferably of between 390 mbar and 1012.9 mbar and more preferably still of between 490 mbar and 1012.9 mbar.

Within the meaning of the present invention, the term “reduced oxygen partial pressure” is understood to mean an oxygen O₂ partial pressure of between 0.1 mbar and 10 mbar, preferably between 0.5 mbar and 5 mbar and more preferably still of between 0.5 mbar and 2 mbar.

The packaging material used for the present invention can be chosen in particular from packagings based on ethylene/vinyl alcohol (“EVOH”) copolymer, on poly(vinylidene chloride) (“PVDC”), on polyamide (“PA”), on polyacrylonitrile (“PAN”), on poly(glycolic acid) (“PGA”) and/or on high density polyethylene (“HDPE”). The packaging material can also be based on aluminum and/or on another appropriate metal, for example deposited on the external surface of a liner, or a sheet of aluminum and/or of another appropriate metal. The packaging material used can also be chosen from IBC (Intermediate Bulk Containers) containers and metal containers supplied or not supplied with at least one inner liner and/or with at least one epoxy coating. Advantageously, the packaging material can be composed of a combination of at least two of the abovementioned materials.

The overall thickness of the packaging does not play a determining role in the present invention. This is because it can be a thin and flexible material, for example of film or sheet type, the thickness of which does not exceed a few tens of or a few hundred microns, but also a stiffer material in the form of a container having a predetermined shape.

The packaging can advantageously be provided in the form of sachets, liners, bags, barrels or cans of all shapes, dimensions and capacities.

By way of example, the packaging can be a liner or pouch which, for the purpose of the transportation or storage of dianhydrohexitol, may be already present or may be intended to be present in a flexible container, such as an aluminum bag or a big bag (or “Flexible Intermediate Container”=FIC) made of canvas or cloth, or in a rigid container, such as a cardboard box.

The applicant company has in particular obtained excellent results in terms of stability on storage with a “PE+Alu” packaging corresponding to an inner liner made of polyethylene (PE) with a thickness of 100 μm in combination with an outer liner consisting of an aluminum complex (comprising polyethylene with a thickness of 80 μm covered with aluminum with a thickness of 8.5 μm).

A subject matter of the present invention is advantageously a packaged dianhydrohexitol, characterized in that the packaging material comprises at least one aluminum-based layer.

In the present invention, the dianhydrohexitol is hermetically packaged, that is to say that the packaging for example the sachet, the liner, the bag, the barrel, and the like, comprising the dianhydrohexitol is closed, for example by heat sealing or by means of an appropriate tie, so as far as possible to limit and, if possible, so as to eliminate any exchange of gas between the interior of the packaging and the external atmosphere.

The dianhydrohexitols packaged according to the invention can be obtained by employing various types of technologies for placing under vacuum or for rendering inert, in particular, technologies such as those involving vessels inside which a vacuum can be established or those which make possible direct inerting of the packagings and optionally filling under an inert atmosphere. Mention may be made, by way of example, of nitrogen N₂ as inerting gas.

According to a preferred form of the present invention, the equipment for placing under vacuum can be a tunnel vessel sold by Bernhardt. Such an item of equipment is composed of a vessel, the internal absolute pressure of which can be lowered using a vacuum pump, and where, optionally, the internal absolute pressure can subsequently be raised using a system for reinjection of gas, in particular of nitrogen, and of an automatic system, inside the vessel, for hermetically closing the packagings.

The packaging containing the dianhydrohexitols can thus be introduced into the vessel, which is then hermetically closed. The absolute pressure inside the vessel is lowered, thus changing from atmospheric pressure, that is to say from approximately 1013 mbar, to a lower pressure, by way of example to an internal absolute pressure of between 0.1 mbar and 10 mbar. The absolute pressure within the closed chamber can then optionally be raised by introduction of an inert gas, in particular of nitrogen with a purity of greater than or equal to 99.995%. The packaging is then hermetically closed and then removed from the vessel.

Another subject matter of the present invention is thus a process for packaging dianhydrohexitol comprising the introduction of said dianhydrohexitol into a gastight packaging material and then the hermetic closing of said packaging, said process being characterized in that it is carried out in an atmosphere exhibiting an oxygen partial pressure of between 0.1 and 10 mbar, preferably of between 0.5 and 5 mbar and more preferably still of between 0.5 and 2 mbar.

Prior to the hermetic closing of the packaging, the low absolute pressure inside the vessel resulting from being placed under vacuum can be compensated for by the introduction into said vessel of an inert gas. Thus, another subject matter of the present invention is a process for packaging dianhydrohexitol such that the stage of closing the packaging is carried out at a pressure of between 250 and 1013 mbar, preferably between 400 and 1013 mbar and more preferably between 500 and 1013 mbar.

The dianhydrohexitols of the present invention can result from a distillation of a crude reaction product, from a product purified by melt crystallization or crystallization in an aqueous or organic solvent, optionally followed by a melting, from a concentration to dryness of a purified solution of dianhydrohexitols according to the patent EP 1 287 000.

The dianhydrohexitols packaged in accordance with the invention advantageously exhibit a stability on storage at 50° C. and at 80° C. in a dry oven, evaluated according to test A, of greater than 1 month, preferably of greater than 2 months and more preferably still of greater than 3 months.

The stability test A consists in evaluating, in a first step, the pH of a sample of product dissolved, at 40% by weight of dry matter, in osmosed water. Subsequently, 50 g of another sample of this same product are introduced into a packaging material to be tested and the sample is packaged according to the process of the present invention. The packaged product is subsequently placed in a ventilated oven, thermostatically controlled at a temperature of 50° C. or 80° C. Several packagings, filled with the same product, are placed in the oven. After a predetermined period, all of the sample of product is extracted from the packaging materials and is dissolved, at 40% by weight of dry matter, in osmosed water. The pH measurement is carried out on a pHmeter of Radiometer Analytical PHM 220 brand equipped with a combined Ag/AgCl wire electrode of Mettler Toledo brand, calibrated beforehand using pH 7 and 4 buffer solutions.

The dianhydrohexitols packaged in accordance with the invention advantageously exhibit a pH, evaluated according to test A, of greater than or equal to 6.0, preferably of between 6.0 and 8.1, after storage for at least 1 month in a ventilated oven, thermostatically controlled at a temperature of 50° C. or 80° C. This pH of greater than or equal to 6.0 shows an absence of generation of acidity (synonymous with decomposition of the dianhydrohexitols with formation of formic acid) and demonstrates an excellent stability of the dianhydrohexitols packaged in accordance with the invention.

The dianhydrohexitols packaged in accordance with the invention advantageously do not exhibit compacting, even after long-term storage at high temperature. The dianhydrohexitols packaged in accordance with the invention thus advantageously exhibit, at the outlet of their packaging, a flow profile, as determined according to the test B, equivalent to that which these same dianhydrohexitols exhibited immediately after they had been manufactured.

The flow test B is carried out using the VS 1000 laboratory siever sold by Retsch, according to the method recommended in the directions for use of said siever. In order to carry out the flow test B, said siever is equipped with a sieving tower composed of 3 sieves with a diameter of 20 cm, the mesh sizes of which are respectively 20 000 μm, 5000 μm and 1000 μm (the sieves are placed from the top downward, from the widest mesh size down to the narrowest mesh size). Briefly, the test B consists in introducing a test portion of 200 g of product at the top of the sieving tower and in starting the siever in continuous mode, at a vibrational amplitude of 50% for 10 minutes. After sieving for 10 minutes, the siever is halted and the amount of product retained on each of the sieves is quantified by weighing. The result of the flow test B is thus expressed as percentage of product retained on each of the sieves, with respect to the total amount of product introduced at the top of the sieving tower. The product does not exhibit compacting after storage if its flow profile (comparison of the percentages of material retained on each sieve) is similar to that of the same product freshly manufactured and thus subjected to a storage period.

The dianhydrohexitols packaged according to the invention or capable of being obtained by the process according to the invention make it possible to produce compositions particularly suited to fields as diversified as nutraceuticals, pharmaceuticals, cosmetics, chemistry, construction materials, paper/board or polymers. Thus, the present invention additionally relates to the use of the dianhydrohexitols packaged according to the invention or obtained by the process according to the invention in the manufacture of derivatives of dianhydrohexitols and of polymers comprising at least one dianhydrohexitol or one derivative of the latter.

An even better understanding of the invention will be obtained with the help of the following examples, which are not meant to be limiting and report only certain embodiments and certain properties which are advantageous of the dianhydrohexitols packaged in accordance with the invention.

### EXAMPLES

### Example 1

### Stability of the Isosorbide in the Solid State as a Function of the Oxygen Partial Pressure in the Packaging

The isosorbide samples tested were as follows:
P: isosorbide with a purity of 99.5% by weight, in the form of flakes;
P pellets: isosorbide with a purity of 99.5% by weight, in the form of pellets (diameter 4 mm and thickness 2 mm).

The samples were packaged in a “PE 30 Alu” packaging corresponding to an inner liner made of polyethylene (PE) with a thickness of 100 μm in combination with an outer liner composed of an aluminum complex (comprising 80 μm of thickness of polyethylene covered with 8.5 μm of thickness of aluminum).

The packaging was carried out in a tunnel vessel sold by Bernhardt. The initial vacuum produced in the tunnel vessel varied from atmospheric pressure (P atm: 1013 mbar at sea level) to a pressure of 5 mbar (initial absolute pressure: column 1, table 1), which corresponds to an oxygen partial pressure (P part O₂, expressed in mbar) of 212 mbar to 0.5 mbar (column 2, table 1). The final absolute pressure inside the vessel, before the hermetic heat sealing of the packaging, was brought back to 1013 mbar by reinjection of nitrogen with a purity of at least 99.995%.

The packaged samples were subjected to the stability test A described above (conditions of the test A: dry oven at 50° C., storage for one or two months). The pHmetry results obtained on conclusion of the stability test A are presented in table 1.

TABLE 1

| | | pH | | | |
|---|---|---|---|---|---|
| Initial absolute | | P | | P pellets | |
| pressure | P part O₂ | 1 | 2 | 1 | 2 |
| (mbar) | (mbar) | month | months | month | months |
| P atm | 212 | 3.1 | === | 4.2 | === |
| 400 | 84 | 3.2 | === | 4.1 | === |
| 180 | 38 | 3.3 | === | 3.9 | === |
| 130 | 27 | 3.5 | === | 4.1 | === |
| 90 | 19 | 3.7 | === | 4.2 | === |
| 23 | 5 | 6.8 | 6.2 | 7.3 | 7.3 |
| 10 | 2 | 6.8 | 6.7 | 7.3 | 7.2 |
| 5 | 1 | 7.0 | 6.6 | 7.2 | 7.3 |

The pH of the samples retained in an environment for which the oxygen partial pressure is less than or equal to 10 mbar, more particularly less than or equal to 5 mbar, remains on the whole stable (pH≧6.0) for at least two months. An analysis carried out after packaging for two months did not demonstrate any presence of peroxides, that is to say of labels of phenomena of oxidation and thus of instability.

All of the samples not packaged in accordance with the invention exhibit, after 1 month of storage, a pH significantly lower than 4.5 and an odor characteristic of formic acid.

This example clearly shows the superiority, in terms of chemical stability, of a dianhydrohexitol packaged according to the invention, in comparison with the conventional processes of packaging, where the oxygen partial pressure inside the packaging is much greater than 10 mbar.

### Example 2

### Stability of the Isosorbide in the Molten State as a Function of the Oxygen Partial Pressure in the Packaging

The isosorbide samples tested were samples of isosorbide with a purity of 99.5% by weight which is maintained at a temperature of approximately 80° C. (P molten). The samples were packaged in a packaging composed of a single liner of an aluminum complex (comprising 80 μm of thickness of polyethylene covered with aluminum with a thickness of 8.5 μm).

The packaging was carried out in a tunnel vessel sold by Bernhardt. The initial vacuum produced in the tunnel vessel varied from atmospheric pressure (P atm) to a pressure of approximately 23 mbar (initial absolute pressure: column 1, table 2), which corresponds to an oxygen partial pressure of 212 mbar to 5 mbar (P part O₂: column 2, table 2). The final absolute pressure inside the vessel, before the hermetic closing of the packaging, was brought back to 1013 mbar by reinjection of nitrogen with a purity of at least 99.995%.

The packaged samples were subjected to the stability test A described above (conditions of test A: dry oven at 80° C., storage for 15 days or for one month). The pHmetry results obtained on conclusion of the stability test A are presented in table 2.

TABLE 2

| | |
|---|---|
| Initial absolute | pH |

| pressure | P part O₂ | 15 | 1 |
|---|---|---|---|
| (mbar) | (mbar) | days | month |
| P atm | 212 | 3.1 | === |
| 23 | 5 | 6.3 | 6.0 |

The pH of the samples stored in an environment for which the oxygen partial pressure is less than 10 mbar, more particularly of the order of 5 mbar, remains on the whole stable (pH 6.0) for at least one month.

On the other hand, the samples not packaged in accordance with the invention exhibit, after storage for 15 days, a pH significantly lower than 3.5 and an odor characteristic of formic acid.

This example clearly shows the superiority, in terms of chemical stability, of a molten dianhydrohexitol packaged according to the invention, in comparison with the conventional processes of packaging, where the oxygen partial pressure inside the packaging is much greater than 10 mbar.

### Example 3

### Stability of the Isosorbide as a Function of the Nature of the Packaging

The isosorbide samples tested were samples of isosorbide with a purity of 99.5% by weight, comprising 0.2% by weight of water and in the form of flakes.

The samples are packaged in different packagings, the natures of which are as follows:
PE+Alu=packaging composed of an inner liner made of polyethylene (PE) with a thickness of 100 μm and of an outer liner composed of an aluminum complex (comprising 80 μm of thickness of polyethylene covered with 8.5 μm of thickness of aluminum)
Alu alone=packaging composed of a single liner of an aluminum complex (comprising 80 μm of thickness of polyethylene covered with 8.5 μm of thickness of aluminum)
Ca+Alu=packaging composed of a first carbon-additivated polyethylene liner with a thickness of 150 μm and of a second outer liner composed of an aluminum complex (comprising 80 μm of thickness of polyethylene covered with 8.5 μm of thickness of aluminum)
TyveK+Alu=packaging composed of a first liner based on high density polyethylene fibers with a thickness of 100 μm and of a second outer liner composed of an aluminum complex (comprising 80 μm of thickness of polyethylene covered with 8.5 μm of thickness of aluminum).

The packaging was carried out in a tunnel vessel sold by Bernhardt. The initial vacuum produced in the tunnel vessel varied from atmospheric pressure (P atm) to a pressure of 23 mbar, which corresponds to an oxygen partial pressure of 212 mbar to 5 mbar. The final absolute pressure inside the vessel, before the hermetic closing of the packaging, was brought back to 1013 mbar by reinjection of nitrogen with a purity of at least 99.995%.

As regards the sample “PE +Alu/stream of nitrogen”, it was subjected to a specific packaging:
the packaging was carried out in a standard environment (absolute pressure equal to atmospheric pressure, oxygen partial pressure of the order of 212 mbar),
the filling of the packaging was carried out under a continuous stream of nitrogen, so as to drive off as much ambient air as possible from the inside of the packaging,
the continuous stream of nitrogen was only halted after the packaging had been hermetically closed.

The packaged samples were subjected to the stability test A described above (conditions of the test A: dry oven at 50° C., storage for 1, 2 or 3 months). The pHmetry results obtained on conclusion of the stability test A are presented in table 3.

TABLE 3

| | P part O₂ | pH | | |
|---|---|---|---|---|
| Packaging | (mbar) | 1 month | 2 months | 3 months |
| Alu alone | 212 | 3.5 | === | === |
| Alu alone | 101 | 3.7 | === | === |
| Alu alone | 50 | 3.4 | === | === |
| Alu alone | 5 | 7.5 | 7.4 | 7.4 |
| PE + Alu | 212 | 3.2 | === | === |
| PE + Alu | 101 | 3.3 | === | === |
| PE + Alu | 50 | 3.6 | === | === |
| PE + Alu | 5 | 7.6 | 7.5 | 7.4 |
| PE + Alu | Stream of | 3.2 | === | === |
| | nitrogen | | | |
| Ca + Alu | 212 | 3.2 | === | === |
| Ca + Alu | 101 | 3.4 | === | === |
| Ca + Alu | 5 | 7.5 | 7.3 | 7.4 |
| TyveK + Alu | 212 | 3.3 | === | === |
| TyveK + Alu | 101 | 3.4 | === | === |
| TyveK + Alu | 5 | 7.6 | 7.5 | 7.5 |

This example clearly demonstrates that any type of packaging can be used provided that it is gastight. Thus, provided that the samples are packaged so that the inside of the packaging exhibits an oxygen O₂ partial pressure of less than or equal to 10 mbar, in particular of the order of 5 mbar, they remain stable at high temperature and over a period of at least 3 months.

This example also demonstrates that the filling of a packaging with dianhydrohexitols while continuously flushing with nitrogen does not make it possible to retain the chemical stability of the dianhydrohexitols over a lengthy period of time (PE+Alu/Stream of nitrogen).

### Example 4

### Compacting of the Isosorbide as a Function of the Pressure within the Packaging

The samples tested were samples of isosorbide P with a purity of 99.5% by weight, in the form of flakes (initial pH=7.0).

The natures of the packagings were as follows:
PE+Alu=packaging composed of an inner liner made of polyethylene (PE) with a thickness of 100 μm and of an outer liner composed of an aluminum complex (comprising 80 μm of thickness of polyethylene covered with 8.5 μm of thickness of aluminum), or
Alu alone=packaging composed of a single liner of an aluminum complex (comprising 80 μm of thickness of polyethylene covered with 8.5 μm of thickness of aluminum).

The packaging was carried out in a tunnel vessel sold by Bernhardt. The initial absolute pressure in the tunnel vessel (initial P absolute) was lowered to a pressure of 24 mbar, which corresponds to an oxygen partial pressure of approximately 5 mbar. The final absolute pressure inside the vessel (final P absolute), before the hermetic heat sealing of the packaging, was optionally raised (up to 500 or 1013 mbar) by reinjection of nitrogen with a purity of at least 99.995%.

The packaged samples were stored in a dry oven at 50° C. over a period of 1 or 2 months. The results of the stability test A and the flow test B obtained on conclusion of the storage are respectively presented in tables 4 and 5 below.

TABLE 4
(Test A)

| | | |
|---|---|---|
| Initial P | Final P | |
| absolute | absolute | pH |

| Packaging | (mbar) | (mbar) | 1 month | 2 months |
|---|---|---|---|---|
| PE + Alu | 24 | 24 | 6.8 | 6.9 |
| Alu alone | 24 | 24 | 6.7 | 6.8 |
| PE + Alu | 24 | 500 | 6.7 | 6.8 |
| Alu alone | 24 | 500 | 6.7 | 6.7 |
| PE + Alu | 24 | 1013 | 6.6 | 6.7 |
| Alu alone | 24 | 1013 | 6.9 | 6.8 |

TABLE 5
(Test B)

| | | | % of material retained after | | | |
|---|---|---|---|---|---|---|
| | Initial | Final | storage for 2 months | | | |
| | P | P | Sieve of mesh size (μm): | | | |
| | absolute | absolute | | | | Not |
| Packaging | (mbar) | (mbar) | 20 000 | 5000 | 1000 | retained |
| Isosorbide | === | === | 0 | 20.3 | 77.2 | 2.5 |
| P at t = 0 | | | | | | |
| PE + Alu | 24 | 24 | 95.6 | 4.4 | 0 | 0 |
| Alu alone | 24 | 24 | 93.7 | 6.3 | 0 | 0 |
| PE + Alu | 24 | 500 | 0 | 25.3 | 73.1 | 1.6 |
| Alu alone | 24 | 500 | 0 | 24.2 | 74.5 | 1.3 |
| PE + Alu | 24 | 1013 | 0 | 20.5 | 77.0 | 2.5 |
| Alu alone | 24 | 1013 | 0 | 20.3 | 77.3 | 2.4 |

This example clearly demonstrates that the samples packaged according to a preferred form of the invention, that is to say with a reduced oxygen partial pressure and an absolute pressure inside the packaging of greater than 250 mbar, preferably of greater than or equal to 500 mbar, following reinjection of nitrogen, remain stable at high temperature and over a period of at least 2 months and do not compact. On the other hand, the samples packaged without reinjection of nitrogen exhibit significant compacting after storage for 2 months, while remaining stable.

1-7. (canceled)

8. A dianhydrohexitol packaged in a gastight packaging material, wherein the oxygen O₂ partial pressure inside the packaging is between 0.1 mbar and 10 mbar.

9. The packaged dianhydrohexitol as claimed in claim 8, wherein the absolute pressure inside the packaging is greater than or equal to 250 mbar.

10. The packaged dianhydrohexitol as claimed in claim 8, wherein the nitrogen N₂ partial pressure inside the packaging is between 240 and 1012.9 mbar.

11. The packaged dianhydrohexitol as claimed in claim 8, wherein the packaging material comprises at least one aluminum-based layer.

12. The packaged dianhydrohexitol as claimed in claim 8, wherein the dianhydrohexitol comprises isosorbide or is essentially composed of isosorbide.

13. A process for packaging dianhydrohexitol comprising the introduction of said dianhydrohexitol into a gastight packaging material and then the hermetic closing of said packaging, wherein said process is carried out in an atmosphere exhibiting an oxygen partial pressure of between 0.1 mbar and 10 mbar.

14. The process for packaging dianhydrohexitol as claimed in claim 13, wherein the stage of closing the packaging is carried out at an absolute pressure of greater than or equal to 250 mbar.

15. The packaged dianhydrohexitol as claimed in claim 8, wherein the oxygen O₂ partial pressure inside the packaging is between 0.5 mbar and 5 mbar.

16. The packaged dianhydrohexitol as claimed in claim 8, wherein the oxygen O₂ partial pressure inside the packaging is between 0.5 mbar and 2 mbar.

17. The packaged dianhydrohexitol as claimed in claim 8, wherein the absolute pressure inside the packaging is greater than or equal to 400 mbar.

18. The packaged dianhydrohexitol as claimed in claim 8, wherein the absolute pressure inside the packaging is greater than or equal to 500 mbar.

19. The packaged dianhydrohexitol as claimed in claim 8, wherein the nitrogen N₂ partial pressure inside the packaging is between 390 mbar and 1012.9 mbar.

20. The packaged dianhydrohexitol as claimed in claim 8, wherein the nitrogen N₂ partial pressure inside the packaging is between 490 mbar and 1012.9 mbar.

21. The process as claimed in claim 13, wherein said process is carried out in an atmosphere exhibiting an oxygen partial pressure of between 0.5 mbar and 5 mbar.

22. The process as claimed in claim 13, wherein said process is carried out in an atmosphere exhibiting an oxygen partial pressure of between 0.5 mbar and 2 mbar.

23. The process as claimed in claim 13, wherein the stage of closing the packaging is carried out at an absolute pressure of greater than or equal to 400 mbar.

24. The process as claimed in claim 13, wherein the stage of closing the packaging is carried out at an absolute pressure of greater than or equal to 500 mbar.
